(19) United States
(12) Patent Application Publication
(10) Pub. No.: US 2019/0191970 A1
(43) Pub. Date: Jun. 27, 2019

(54) HANDHELD VACUUM DEVICE WITH CAMERA AND ILLUMINATION

(71) Applicant: Maurice A. Greene, Manassas, VA (US)

(72) Inventor: Maurice A. Greene, Manassas, VA (US)

(21) Appl. No.: 15/854,146

(22) Filed: Dec. 26, 2017

(51) Int. Cl. A61B 1/00; A61B 1/05; A61B 1/06; A61M 1/00; A61B 17/24

(52) U.S. Cl. CPC A61B 1/00094; A61B 1/00091; A61B 1/05; A61B 17/24; A61M 1/0056; A61B 1/00045; A61B 1/0684

(57) ABSTRACT

The handheld vacuum device disclosed herein comprises, at least, an air pump, a digital camera, and an illumination source. The device uses negative pressure to withdraw foreign objects from areas otherwise difficult to access and visualize, and is preferably used in removing tonsilloliths from the tonsillar crypts of a user. Negative pressure is preferred to prevent dissemination of foreign material down a user's throat and avoid causing additional trauma to the tonsillar crypts. The design disclosed herein further benefits from camera-assisted visualization and illumination to aid in removal of tonsilloliths further down a user's throat and not otherwise visible by unassisted visualization. The endoscopic camera is intended to be a commonly available design that interfaces with standard smart phone devices to display the images acquired through the camera lens.

### PRIORITY NOTICE

The present application is a non-provisional utility application and makes a claim of priority under 35 U.S.C. § 119(e) to prior provisional application 62/444,673 filed in the United States Patent and Trademark Office on Jan. 10, 2017.

### CROSS REFERENCE TO RELATED PATENT APPLICATIONS

The present application makes no reference to any other related filed patent applications.

### STATEMENT REGARDING FEDERAL SPONSORSHIP

No part of this invention was a result of any federally sponsored research.

### TECHNICAL FIELD OF THE INVENTION

The present invention relates in general to handheld vacuum devices, and, more specifically, to a handheld vacuum device comprising, at least, an air pump, a digital camera, and an illumination source.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Tonsilloliths, known in lay terms as tonsil stones, are benign collections of cellular debris that accumulate in the tonsillar crypts of humans. Though common, tonsilloliths may grow large enough to create a sensation of foreign object obstruction or dull pain in the throat of an affected individual. Larger tonsilloliths have also shown a correlation with multiple symptoms including halitosis, known in lay terms as bad breath, throat infection, bad taste, ear ache, and tonsillar swelling. Treatment of such tonsilloliths varies depending on present symptoms and complexity. Many smaller tonsilloliths may resolve spontaneously, while larger tonsilloliths may be removed by an individual using the tip of their tongue. When necessary, oral irrigators or vigorous gargling may assist with removal. Difficult to remove tonsilloliths may respond only to curettage, physical removal by scooping or scraping, or anti-inflammatory compounds.

It is known to have a tonsillolith removal method using oral irrigators to provide positive pressure for physical removal of such foreign objects from tonsillar crypts. Oral irrigators may be known as “dental water jets,” “dental water flossers,” or “waterpiks.” Such designs issue a positive pressure stream of fluid, usually water, that may be directed at a lodged foreign body to aid in its removal. Such designs, though, may produce a too powerful stream of fluid that may rupture the tonsils and lead to further pathology. Such devices may also project a dislodged tonsillolith down the throat of a user, leading to the possibility of disseminating infectious material into the user's body.

It is known to have a tonsillolith removal method using oral curettes to scoop or scrape such foreign objects from tonsillar crypts. Such a method requires physical contact and manipulation between the curette and the tonsillolith, and may result in trauma to the surrounding tonsillar crypt. Such devices also may not allow for complete removal of smaller tonsilloliths, necessitating the subsequent use of a second method of removal. Curettage may be combined with local excision, physical cutting and removal, though this combination still may not allow for complete removal of smaller tonsilloliths.

It is known to have a tonsillolith removal method using positive pressure or curettage in combination with a visualization device to aid in dislodging tonsilloliths further down a user's throat that are not otherwise visible by unassisted visualization. Such visualization devices may be known as “dental mirrors” or “dental mouth mirrors,” and may permit a user to see further down into the throat due to specular reflection, though the process of holding the device in a second hand of the user may itself block the image reflected by the mirror. Such a device also may not provide illumination to the viewing area, further limiting the usefulness of the reflected image.

There is a need in the art for a tonsillolith removal method using negative pressure to prevent dissemination of foreign material down a user's throat and avoid causing additional trauma to the tonsillar crypt. Such a design may further benefit from camera-assisted visualization and illumination to aid in removal of tonsilloliths further down a user's throat and not otherwise visible by unassisted visualization.

It is to these ends that the present invention has been developed.

### BRIEF SUMMARY OF THE INVENTION

To minimize the limitations in the prior art, and to minimize other limitations that will be apparent upon reading and understanding the present specification, the present invention describes a handheld vacuum device comprising, at least, an air pump, a digital camera, and an illumination source.

It is an objective of the present invention to provide a handheld vacuum device comprising, at least, an air pump, a digital camera, and an illumination source to facilitate negative pressure removal of tonsilloliths from a user's tonsillar crypts.

It is another objective of the present invention to provide a handheld vacuum device comprising a negative pressure air pump allowing for gentle suction of tonsilloliths from a user's tonsillar crypts to reduce or prevent injury to the surrounding areas.

It is another objective of the present invention to provide a handheld vacuum device comprising a commonly available camera device for ease of use and reduced cost of maintenance.

It is another objective of the present invention to provide a handheld vacuum device comprising a camera device with a built-in smart phone interface for ease of use.

It is another objective of the present invention to provide a handheld vacuum device comprising a light-emitting diode (LED) illumination source for ease of use, minimization of size and complexity, and longevity of the light source.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates an overview of a handheld vacuum device.

FIG. 2 illustrates an overview of Embodiment 001 of a handheld vacuum device.

FIG. 3 illustrates an exploded view of a handheld vacuum device.

FIG. 4 illustrates an overview of a handheld vacuum device comprising, at least, an air pump, a digital camera, and an illumination source.

FIG. 5 illustrates an overview of Embodiment 002 of a handheld vacuum device.

FIG. 6 illustrates an overview of a handheld vacuum device.

FIG. 7 illustrates an overview of a handheld vacuum device.

FIG. 8 illustrates a preferred air pump for use in a handheld vacuum device.

FIG. 9 illustrates a preferred endoscope for use in a handheld vacuum device.

FIG. 10 illustrates a preferred endoscope for use in a handheld vacuum device.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates generally to handheld vacuum devices and, more specifically, to a handheld device for removing tonsilloliths from the tonsillar crypts of a user. Such a device comprises, at least, an air pump, a digital camera, and an illumination source.

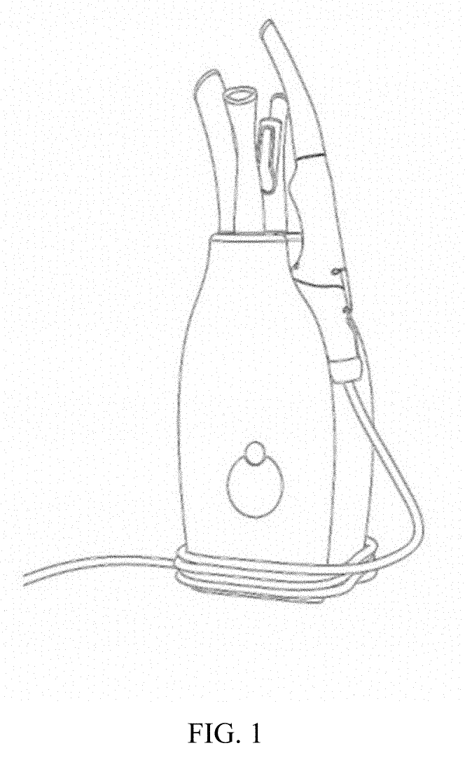

FIG. 1 illustrates an overview of a handheld vacuum device as contemplated by the present disclosure. The figure illustrates, generally, a stored form of said device wherein a handpiece, which may be comprised of a housing body and a catch tank, may be removably stored on an air pump housing, and wherein a plurality of nozzle attachments may be removably stored in said air pump housing.

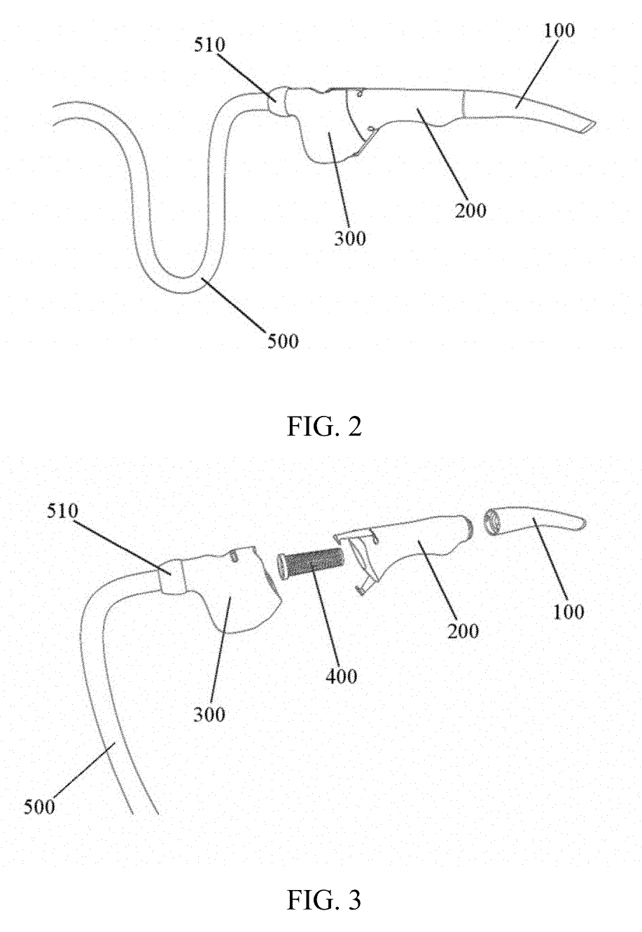

FIG. 2 illustrates an overview of Embodiment 001 of a handheld vacuum device identifying a nozzle attachment 100, a housing body 200, a catch tank 300, a vacuum tube 500, and a vacuum tube attachment 510. The housing body 200 and catch tank 300 may comprise a handpiece to which a plurality of interchangeable nozzle attachments 100 may be removably attached. The housing body 200 may comprise an LED board that may provide illumination within the handpiece itself. Such illumination within the handpiece may be transmitted through the plurality of nozzle attachments 100 by fiber-optic principles. A vacuum force may be transferred through the vacuum tube 500 into the handpiece, and the vacuum tube 500 may be permanently or removably attached to the handpiece by the vacuum tube attachment 510. The vacuum tube 500 may comprise a length between 300 millimeters (mm) and 1,500 mm, though is preferably 900 mm in length. The vacuum tube attachment 510 may exert sufficient force on the vacuum tube 500 to prevent it from coming loose when vacuum force is applied to the system.

The catch tank 300 may contain a tubular internal orifice through which to transmit vacuum force brought by the vacuum tube 500. Such vacuum force may then pass into a tubular internal orifice in the housing body 200. From here the vacuum force may be passed through a plurality of nozzle attachments 100, where such vacuum force is then exerted from a tip end of a nozzle attachment 100.

FIG. 3 illustrates an exploded view of a handheld vacuum device identifying a nozzle attachment 100, a housing body 200, a catch tank 300, a vacuum tube 500, and a vacuum tube attachment 510. Also illustrated is a filter 400. Such a filter may be placed into the tubular internal orifice of the housing body 200 or the catch tank 300 to prevent the passage of tonsilloliths through the handpiece and into the vacuum tube 500.

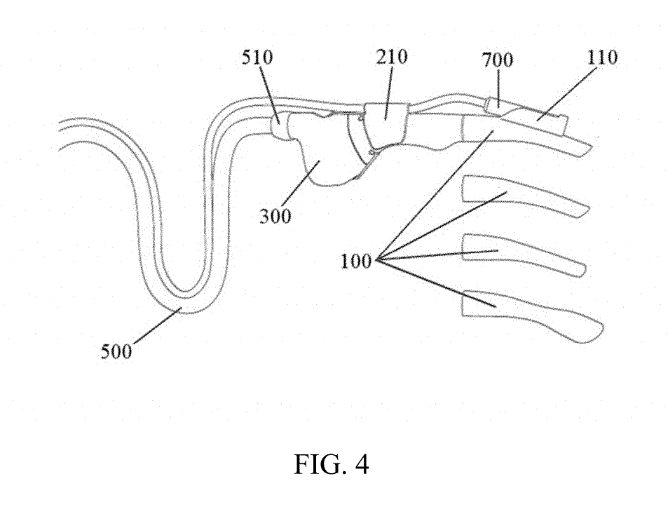

FIG. 4 illustrates an overview of a handheld vacuum device comprising, at least, an air pump, a digital camera, and an illumination source. The figure identifies a plurality of nozzle attachments 100, a catch tank 300, a vacuum tube 500, and a vacuum tube attachment 510. Also illustrated are a camera receiver 110, cable receiver 210, and endoscope 700. The endoscope 700 may comprise any commonly available compatible device with smart phone interface for ease of use and reduced cost of maintenance. The endoscope 700 preferably comprises a waterproof endoscope camera with built-in LED lighting and a universal serial bus (USB) connection. The endoscope head may have a diameter between 3 mm and 10 mm, though the diameter is preferably 5.5 mm, and may comprise a ring of LEDs around a centrally-located camera lens. The brightness of the LEDs may be adjustable via the endoscope hardware, such as a dimmer switch, or via the endoscope software, such as a smart phone application selection. The USB connection may interface with a standard smart phone camera to display the images captured by the camera lens either through the smart phone software or proprietary endoscope software.

The camera receiver 110 may be permanently or removably attached to a plurality of nozzle attachments 100, and may receive the endoscope head of the endoscope 700 to hold it in place and aid in single-handed use of the device. The cable receiver 210 may be permanently or removably attached to the housing body 200, and may receive the endoscope cable of the endoscope 700 to hold it in place and aid in single-handed use of the device.

The plurality of nozzle attachments 100 may comprise hard plastic tubes of varying widths between 25 mm and 250 mm long, though preferably 100 mm long. The varying widths of the plurality of nozzle attachments 100 may be used to adjust the vacuum force through the tip end of the nozzle attachment.

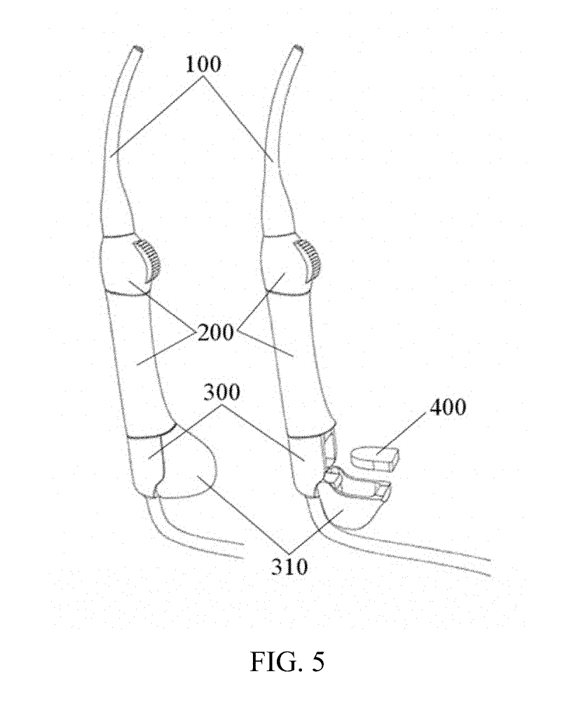

FIG. 5 illustrates an overview of Embodiment 002 of a handheld vacuum device identifying a nozzle attachment 100, a housing body 200, a catch tank 300, a catch tank door 310, and a filter 400. The catch tank door 310 may be removably attached to the catch tank 300 via a hinge mechanism or other appropriate means. The catch tank door 310 may be opened to allow removal of vacuumed tonsilloliths, or installation or removal of the filter 400. The filter 400 may be placed into the tubular internal orifice of the catch tank 300 to prevent the passage of tonsilloliths through the handpiece and into the vacuum tube 500.

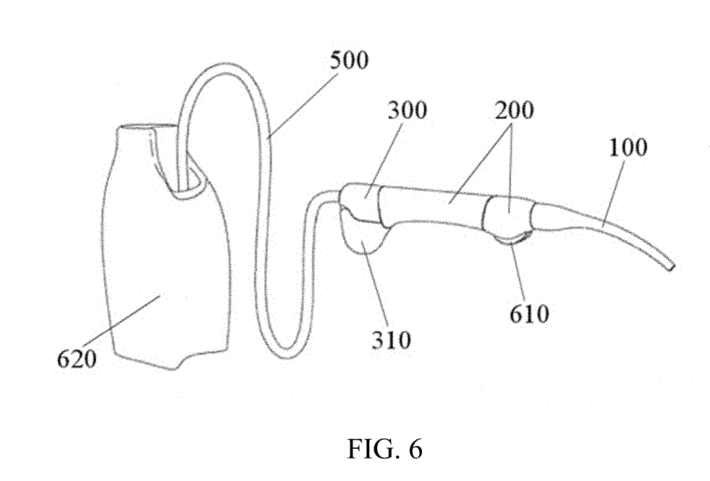

FIG. 6 illustrates an overview of a handheld vacuum device identifying a nozzle attachment 100, a housing body 200, a catch tank 300, a catch tank door 310, a vacuum tube 500, an air pump toggle switch 610, and an air pump housing 620. The air pump toggle switch 610 may comprise any appropriate switch, though preferably is an adjustable switch commonly used to toggle between on, off, and various power settings. Such various power settings may be used to adjust the power applied to an air pump 600 and, thus, the vacuum force provided by an air pump 600. The air pump housing switch 620 may comprise an ergonomic design so that it may be activated without difficulty while the user grips the handpiece.

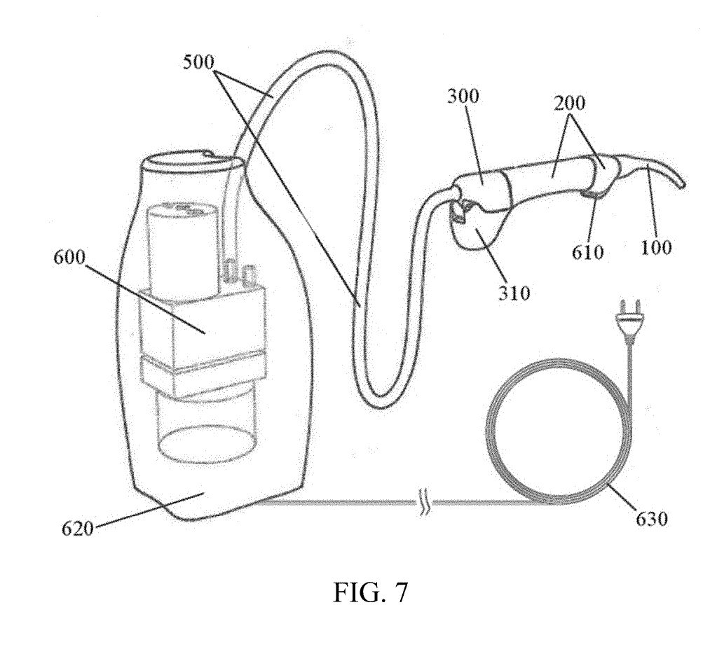

FIG. 7 illustrates an overview of a handheld vacuum device identifying a nozzle attachment 100, a housing body 200, a catch tank 300, a catch tank door 310, a vacuum tube 500, an air pump 600, an air pump toggle switch 610, an air pump housing 620, and a 12-volt power adapter 630. The air pump housing 620 may comprise any hard plastic housing appropriate to isolate and protect the air pump 600, while also providing storage options for the handpiece and the plurality of nozzle attachments 100. The air pump housing 620 may further comprise a 12-volt connector facilitating an electrical connection between the air pump 600 and the 12-volt power adapter 630. The air pump housing 620 may alternatively comprise an opening through which a 12-volt power adapter 630 may pass to facilitate an electrical connection directly to the air pump 600.

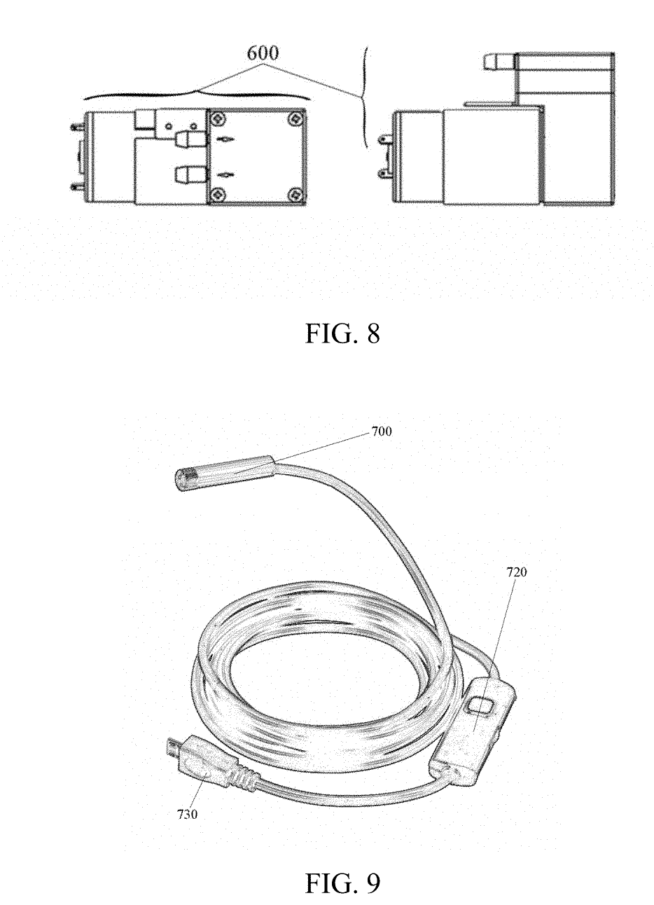

FIG. 8 illustrates a preferred air pump 600 for use in a handheld vacuum device. The air pump 600 may comprise any commonly available compatible air pump, though preferably comprises a D2028 air pump or its equivalent. Such an air pump 600 may produce an effective vacuum force up to 16 millimeters of mercury (mmHg) and may generate an air pressure up to 32 pounds per square inch (psi). The air pump 600 may be powered by any 12-volt alternating current (AC) power source, and may have a normal operating temperature between 32 degrees Fahrenheit (° F.) and 120° F. These specifications are not intended to be limiting, and any appropriate air pump 600 may be substituted into the present device without deviating from the spirit of the invention.

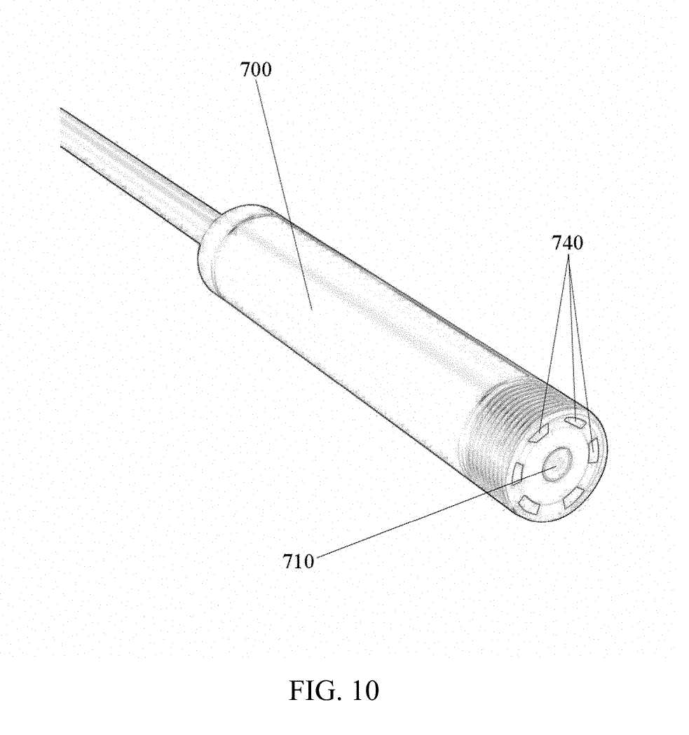

FIGS. 9 and 10 illustrate a preferred endoscope for use in a handheld vacuum device identifying an endoscope 700, a camera 710, a camera toggle switch 720, a USB connector 730, and a plurality of LEDs 740.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A handheld vacuum device, comprising:
a handpiece;
a plurality of nozzle attachments;
an endoscope device;
an air pump; and
a vacuum tube;
wherein said handpiece comprises a housing body and a catch tank;
wherein said endoscope device comprises a camera and a plurality of LEDs;
wherein said air pump is connected to provide vacuum force; and
wherein said vacuum tube connects said air pump to said handpiece.

2. The invention of claim 1, further comprising:
a vacuum tube attachment;
wherein said vacuum tube attachment attaches said vacuum tube to said handpiece.

3. The invention of claim 2, further comprising:
a filter;
wherein said filter is installed in said handpiece to prevent a foreign body from entering said vacuum tube.

4. The invention of claim 3, further comprising:
a plurality of camera receivers;
wherein said camera receivers are attached to said nozzle attachments; and
wherein said camera receivers are removably attached to a head of said endoscope device.

5. The invention of claim 4, further comprising:
a cable receiver;
wherein said cable receiver is removably attached to a cable of said endoscope device.

6. The invention of claim 5, further comprising:
a catch tank door;
wherein said catch tank door is removably attached to said catch tank.

7. The invention of claim 6,
wherein said endoscope device is removably attached to a smart phone; and
wherein said smart phone may display images transmitted from a camera of said endoscope device.
